(19) United States
(12) Patent Application Publication
(10) Pub. No.: US 2024/0016750 A1
(43) Pub. Date: Jan. 18, 2024

(54) FORMULATION CONTAINING IBUPROFEN

(71) Applicant: RECKITT BENCKISER HEALTH LIMITED, Berkshire (GB)

(72) Inventors: Anupam A K DAS, Humberside (GB); Matthew Edward Anthony MCGIRR, Humberside (GB)

(73) Assignee: RECKITT BENCKISER HEALTH LIMITED, Berkshire (GB)

(21) Appl. No.: 17/999,061

(22) PCT Filed: May 21, 2021
(86) PCT No.: PCT/GB2021/051241

§ 371 Date: Nov. 17, 2022

(30) Foreign Application Priority Data
May 21, 2020 (GB) 2007619.6

(51) Int. Cl. A61K 9/48; A61K 31/192; A61K 47/26

(52) U.S. Cl. CPC A61K 9/4866; A61K 31/192; A61K 47/26

(57) ABSTRACT

The present invention relates to a composition for encapsulation in a soft gelatin shell which comprises ibuprofen, one or more polyoxysorbitan esters and a base wherein the weight ratio of the one or more polyoxysorbitan esters to ibuprofen is from 0.1:1-0.9:1, the weight ratio of the base to ibuprofen is from 1:6-1:20 and the weight ratio of the one or more polyoxysorbitan esters to base is 1:1-13:1 and wherein the ibuprofen is present at an amount of 50%-65% w/w.

The present invention is directed to a composition that comprises ibuprofen. In particular, the present invention is directed to a composition that comprises ibuprofen and is suitable for encapsulation in a soft gelatin capsule.

Soft gelatin capsules offer a number of advantages over tablets and caplets as a pharmaceutical dosage form. They are easily digested and dissolve quickly in the stomach thus allowing quicker onset of the desired pharmaceutical effect. Soft gelatin capsules are also very useful for administering poorly soluble or poorly absorbed active pharmaceutical ingredients (API). Soft gelatin capsules are also useful for protecting APIs from light and oxygen thus improving their stability.

Soft gelatin capsules which contain a pain killer, such as ibuprofen, are known in the art and are commercially available. These capsules contain the standard dose of 200 mg or 400 mg of ibuprofen.

U.S. Pat. No. 5,376,688 describes soft and hard gelatin capsules containing a fill formulation which comprises a diethylene glycol monoethyl ether and a polyglycerol oleate. U.S. Pat. No. 5,912,011 discloses a solvent system for encapsulation in soft and hard gelatin capsules. WO 88/02625 discloses a solvent system to enhance the solubility of APIs. WO 2005/123133 discloses a solvent system for APIs which comprises 15-50% by weight of polyethylene glycol.

However, while the soft gelatin capsules that are currently available are increasingly attractive to consumers one aspect that could be improved is the size of the capsule. A smaller dosage form is easier to swallow and provides a better experience for consumers. The present invention provides a soft-gelatin capsule that is significantly smaller than current capsules but maintains or improves on the therapeutic profile of existing capsules. The soft-gelatin capsule contains a stable highly concentrated solution of ibuprofen that is able to provide an effective dose.

According to an aspect of the present invention there is provided a composition for encapsulation in a soft gelatin shell which comprises ibuprofen, one or more polyoxysorbitan esters and a base wherein the weight ratio of the one or more polyoxysorbitan esters to ibuprofen is from 0.1:1-0.9:1, the weight ratio of the base to ibuprofen is from 1:6-1:20 and the weight ratio of the one or more polyoxysorbitan esters to base is 1:1-13:1 and wherein the ibuprofen is present at an amount of 50%-65% w/w.

Preferably the weight ratio of the one or more polyoxysorbitan esters to ibuprofen is 0.4:1-0.8:1. More preferably the weight ratio of the one or more polyoxysorbitan esters to ibuprofen is 0.5:1-0.7:1.

Preferably the weight ratio of base to ibuprofen is 1:8-1:15. More preferably the weight ratio of base to ibuprofen is 1:9-1:11.5.

Preferably the weight ratio of the one or more polyoxysorbitan esters to base is 3:1-9:1. More preferably the weight ratio of the one or more polyoxysorbitan esters to base is 5.5:1-7.75:1.

Preferably the weight ratio of the one or more polyoxysorbitan esters to ibuprofen is 0.5:1-0.7:1, the weight ratio of base to ibuprofen is 1:9-1:11.5 and the weight ratio of the one or more polyoxysorbitan esters to base is 5.5:1-7.75:1.

The composition can comprise ibuprofen, one or more polyoxysorbitan esters and a base wherein the weight ratio of the ibuprofen:the one or more polyoxysorbitan esters:base is about 6-20:1-13:1.

The composition can comprise ibuprofen, one or more polyoxysorbitan esters and a base wherein the weight ratio of the ibuprofen:the one or more polyoxysorbitan esters:base is about 9-12:5-8:1.

The one or more polyoxysorbitan esters can be selected from the group consisting of polysorbate 20, polysorbate 21, polysorbate 40, polysorbate 60, polysorbate 61, polysorbate 65, polysorbate 80, polysorbate 81, polysorbate 85 or polysorbate 120. A preferred polyoxysorbitan ester is polysorbate 80.

The base can be selected from alkali metal hydroxides (i.e. the metals of Group I of the Periodic Table) particularly sodium and potassium, and alkali earth metal hydroxides (i.e. the metals of Group II of the Periodic Table) particularly calcium and magnesium. Preferred hydroxides are sodium hydroxide and potassium hydroxide. A more preferred hydroxide is potassium hydroxide.

The base can also be selected from carbonate and bicarbonate salts of the alkali and alkali earth metals, i.e. the metals of Group I or Group II of the periodic table.

The base can be selected from amines and amino acids such as ammonia, triethylamine, lysine or arginine.

The composition can further comprise polyethylene glycol in an amount from about 2% to about 25% w/w. Preferably the polyethylene glycol is present in an amount from about 5% to about 10% w/w. More preferably the polyethylene glycol is present in an amount from about 6% to about 7% w/w.

Typically, the polyethylene glycol is a liquid at room temperature. The polyethylene glycol can have a number average molecular weight (Mn) of up to 1000. The polyethylene glycol can have a number average molecular weight (Mn) of from about 400 to about 800. Preferred polyethylene glycols can have a number average molecular weight (Mn) of 200, 300, 400, 600, 800. Preferred polyethylene glycols can have a number average molecular weight (Mn) of 400 or 600.

The composition can comprise a poloxysorbitan ester selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

The composition can further comprise water in an amount from about 2% w/w to about 10% w/w. The water can be present at a level of from 3% w/w to 5.5% w/w.

Typically, the unit dose weight of the formulation can be about 300-420 mg. More typically, the unit dose weight of the formulation can be about 320-400 mg. Even more typically, the unit dose weight of the formulation can be about 340-380 mg. Most typically, the unit dose weight of the formulation can be about 370 mg.

Typically, the unit dose has a fill volume of from 0.3 ml to 0.42 ml.

Alternatively, the unit dose weight of the formulation can be about 600-840 mg. The unit dose weight of the formulation can be about 640-800 mg. The unit dose weight of the formulation can be about 680-760 mg. The unit dose weight of the formulation can be about 740 mg. Typically, the unit dose has a fill volume of from 0.6 ml to 0.84 ml.

The composition can comprise ibuprofen, one or more polyoxysorbitan esters and a hydroxide wherein ibuprofen is present at an amount of 50-65% w/w, the one or more polyoxysorbitan esters is present at an amount of 30-40% w/w, the hydroxide is present at an amount of 3-10% w/w, and the weight ratio of the one or more polyoxysorbitan esters to ibuprofen is 0.5:1-0.7:1, the weight ratio of hydroxide to ibuprofen is 1:9-1:11.5 and the weight ratio of the one or more polyoxysorbitan esters to hydroxide is 5.5:1-7.75:1.

The composition can comprise ibuprofen, one or more polyoxysorbitan esters and a hydroxide wherein ibuprofen is present at an amount of 50-65% w/w, the one or more polyoxysorbitan esters is present at an amount of 30-40% w/w, the hydroxide is present at an amount of 4-7% w/w, and the weight ratio of the one or more polyoxysorbitan esters to ibuprofen is 0.5:1-0.7:1, the weight ratio of hydroxide to ibuprofen is 1:9-1:11.5 and the weight ratio of the one or more polyoxysorbitan esters to hydroxide is 5.5:1-7.75:1.

The composition can comprise ibuprofen, one or more polyoxysorbitan esters, a hydroxide and a polyethylene glycol wherein ibuprofen is present at an amount of 50-65% w/w, the one or more polyoxysorbitan esters is present at an amount of 20-35% w/w, the hydroxide is present at an amount of 3-10% w/w, the polyethylene glycol is present as at an amount of 5-10% w/w and the weight ratio of the one or more polyoxysorbitan esters to ibuprofen is 0.5:1-0.6:1, the weight ratio of hydroxide to ibuprofen is 1:9-1:11 and the weight ratio of the one or more polyoxysorbitan esters to hydroxide is 5:1-6:1.

The composition can comprise ibuprofen, one or more polyoxysorbitan esters, a hydroxide and a polyethylene glycol wherein ibuprofen is present at an amount of 50-65% w/w, the one or more polyoxysorbitan esters is present at an amount of 20-35% w/w, the hydroxide is present at an amount of 4-7% w/w, the polyethylene glycol is present as at an amount of 5-10% w/w and the weight ratio of the one or more polyoxysorbitan esters to ibuprofen is 0.4:1-0.6:1, the weight ratio of hydroxide to ibuprofen is 1:9-1:11 and the weight ratio of the one or more polyoxysorbitan esters to hydroxide is 5:1-6:1.

According to an aspect of the present invention there is provided a composition comprising:
(a) 50-65% w/w ibuprofen;
(b) 25-45% w/w One or more polyoxysorbitan esters;
(c) 3-8% w/w A hydroxide; and
(d) Water
   wherein the volume of the unit dose of the composition is from about 0.3 ml to about 0.42 ml.

Preferably, the composition can comprise:
(a) 50-60% w/w ibuprofen;
(b) 35-40% w/w One or more polyoxysorbitan esters;
(c) 3-8% w/w A hydroxide; and
(d) Water

More preferably, the composition can comprise:
(a) 50-65% w/w ibuprofen;
(b) 5-35% w/w One or more polyoxysorbitan esters;
(c) 5-10% w/w A hydroxide;
(d) 5-25% w/w Polyethylene glycol; and
(e) Water.

The composition can consist essentially of:
(a) 50-65% w/w ibuprofen;
(b) 25-40% w/w One or more polyoxysorbitan esters;
(c) 3-8% w/w A hydroxide; and
(d) Water.

The composition can consist essentially of:
(a) 52-55% w/w ibuprofen;
(b) 35-40% w/w One or more polyoxysorbitan esters;
(c) 3-5% w/w A hydroxide; and
(d) Water.

The composition can consist essentially of:
(a) 50-65% w/w ibuprofen;
(b) 5-35% w/w One or more polyoxysorbitan esters;
(c) 5-10% w/w A hydroxide;
(d) 5-25% w/w Polyethylene glycol; and
(e) Water.

The composition can consist essentially of:
(a) 50-55% w/w ibuprofen;
(b) 30-35% w/w One or more polyoxysorbitan esters;
(c) 5-8% w/w A hydroxide;
(d) 4-8% w/w Polyethylene glycol; and
(e) Water.

The composition can consist essentially of:
(a) 50-65% w/w ibuprofen;
(b) 25-40% w/w One or more polyoxysorbitan esters;
(c) 3-8% w/w A hydroxide; and
(d) Water
   wherein the volume of the unit dose of the composition is from about 0.3 ml to about 0.42 ml.

The composition can consist essentially of:
(a) 52-55% w/w ibuprofen;
(b) 35-40% w/w One or more polyoxysorbitan esters;
(c) 3-5% w/w A hydroxide; and
(d) Water
   wherein the volume of the unit dose of the composition is from about 0.3 ml to about 0.42 ml.

The composition can consist essentially of:
(a) 50-65% w/w ibuprofen;
(b) 5-35% w/w One or more polyoxysorbitan esters;
(c) 5-10% w/w A hydroxide;
(d) 5-25% w/w Polyethylene glycol; and
(e) Water
   wherein the volume of the unit dose of the composition is from about 0.3 ml to about 0.42 ml.

The composition can consist essentially of:
(a) 50-55% w/w ibuprofen;
(b) 30-35% w/w One or more polyoxysorbitan esters;
(c) 5-8% w/w A hydroxide;
(d) 4-8% w/w Polyethylene glycol; and
(e) Water
   wherein the volume of the unit dose of the composition is from about 0.3 ml to about 0.42 ml.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan ester selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a composition comprising:
(a) 50-65% w/w ibuprofen;
(b) 25-45% w/w One or more polyoxysorbitan esters;
(c) 3-8% w/w A hydroxide; and
(d) Water
   wherein the volume of the unit dose of the composition is from about 0.6 ml to about 0.84 ml.

Preferably, the composition can comprise:
(a) 50-60% w/w ibuprofen;
(b) 35-40% w/w One or more polyoxysorbitan esters;
(c) 3-8% w/w A hydroxide; and
(d) Water.

More preferably, the composition can comprise:
(a) 50-65% w/w ibuprofen;
(b) 5-35% w/w One or more polyoxysorbitan esters;
(c) 5-10% w/w A hydroxide; and
(d) 5-25% w/w Polyethylene glycol.

The composition can consist essentially of:
(a) 50-65% w/w ibuprofen;
(b) 25-40% w/w One or more polyoxysorbitan esters;
(c) 3-8% w/w A hydroxide; and
(d) Water
   wherein the volume of the unit dose of the composition is from about 0.6 ml to about 0.84 ml.

The composition can consist essentially of:
(a) 52-55% w/w ibuprofen;
(b 35-40% w/w One or more polyoxysorbitan esters;
(c) 3-5% w/w A hydroxide; and
(d) Water
   wherein the volume of the unit dose of the composition is from about 0.6 ml to about 0.84 ml.

The composition can consist essentially of:
(a) 50-65% w/w ibuprofen;
(b) 5-35% w/w One or more polyoxysorbitan esters;
(c) 5-10% w/w A hydroxide;
(d) 5-25% w/w Polyethylene glycol; and
(e) Water
   wherein the volume of the unit dose of the composition is from about 0.6 ml to about 0.84 ml.

The composition can consist essentially of:
(a) 50-55% w/w ibuprofen;
(b) 30-35% w/w One or more polyoxysorbitan esters;
(c) 5-8% w/w A hydroxide;
(d) 4-8% w/w Polyethylene glycol; and
(e) Water
   wherein the volume of the unit dose of the composition is from about 0.6 ml to about 0.84 ml.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan ester selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a composition comprising:
(a) 50-65% w/w ibuprofen;
(b) 25-45% w/w One or more polyoxysorbitan esters;
(c) 3-8% w/w A hydroxide; and
(d) Water
   wherein the unit dose weight of the formulation can be about 300-420 mg.

Typically, the unit dose weight of the formulation can be about 320-400 mg. More typically, the unit dose weight of the formulation can be about 340-380 mg. Most typically, the unit dose weight of the formulation can be about 360-370 mg.

Preferably, the composition can comprise:
(a) 50-60% w/w ibuprofen;
(b) 35-40% w/w One or more polyoxysorbitan esters;
(c) 3-8% w/w A hydroxide; and
(d) Water.

The composition can comprise:
(a) 50-65% w/w ibuprofen;
(b) 5-35% w/w One or more polyoxysorbitan esters;
(c) 5-10% w/w A hydroxide;
(d) 5-25% w/w Polyethylene glycol; and
(e) Water.
   wherein the unit dose weight of the formulation can be about 300-420 mg.

Typically, the unit dose weight of the formulation can be about 320-400 mg. More typically, the unit dose weight of the formulation can be about 340-380 mg. Most typically, the unit dose weight of the formulation can be about 360-370 mg.

The composition can consist essentially of:
(a) 50-65% w/w ibuprofen;
(b) 25-40% w/w One or more polyoxysorbitan esters;
(c) 3-8% w/w A hydroxide; and
(d) Water
   wherein the unit dose weight of the formulation can be about 300-420 mg.

Typically, the unit dose weight of the formulation can be about 320-400 mg. More typically, the unit dose weight of the formulation can be about 340-380 mg. Most typically, the unit dose weight of the formulation can be about 360-370 mg.

Preferably the composition can consist essentially of:
(a) 52-55% w/w ibuprofen;
(b) 35-40% w/w One or more polyoxysorbitan esters;
(c) 3-5% w/w A hydroxide; and
(d) Water.

The composition can consist essentially of:
(a) 50-65% w/w ibuprofen;
(b) 5-35% w/w One or more polyoxysorbitan esters;
(c) 5-10% w/w A hydroxide;
(d) 5-25% w/w Polyethylene glycol; and
(e) Water
   wherein the unit dose weight of the formulation can be about 300-420 mg.

Typically, the unit dose weight of the formulation can be about 320-400 mg. More typically, the unit dose weight of the formulation can be about 340-380 mg. Most typically, the unit dose weight of the formulation can be about 360-370 mg.

The composition can consist essentially of:
(a) 50-55% w/w ibuprofen;
(b) 30-35% w/w One or more polyoxysorbitan esters;
(c) 5-8% w/w A hydroxide;
(d) 4-8% w/w Polyethylene glycol; and
(e) Water
wherein the unit dose weight of the formulation can be about 300-420 mg.

Typically, the unit dose weight of the formulation can be about 320-400 mg. More typically, the unit dose weight of the formulation can be about 340-380 mg. Most typically, the unit dose weight of the formulation can be about 360-370 mg.

The composition can consist essentially of:
(a) 50-65% w/w ibuprofen;
(b) 25-40% w/w One or more polyoxysorbitan esters;
(c) 3-8% w/w A hydroxide; and
(d) Water
wherein the unit dose weight of the formulation can be about 300-420 mg.

Typically, the unit dose weight of the formulation can be about 320-400 mg. More typically, the unit dose weight of the formulation can be about 340-380 mg. Most typically, the unit dose weight of the formulation can be about 360-370 mg.

Preferably, the composition can consist essentially of:
(a) 52-55% w/w ibuprofen;
(b) 35-40% w/w One or more polyoxysorbitan esters;
(c) 3-5% w/w A hydroxide; and
(d) Water

The composition can consist essentially of:
(a) 50-65% w/w ibuprofen;
(b) 5-35% w/w One or more polyoxysorbitan esters;
(c) 5-10% w/w A hydroxide;
(d) 5-25% w/w Polyethylene glycol; and
(e) Water
   wherein the unit dose weight of the formulation can be about 300-420 mg.

Typically, the unit dose weight of the formulation can be about 320-400 mg. More typically, the unit dose weight of the formulation can be about 340-380 mg. Most typically, the unit dose weight of the formulation can be about 360-370 mg.

The composition can consist essentially of:
(a) 50-55% w/w ibuprofen;
(b) 30-35% w/w One or more polyoxysorbitan esters;
(c) 5-8% w/w A hydroxide;
(d) 4-8% w/w Polyethylene glycol; and
(e) Water
   wherein the unit dose weight of the formulation can be about 300-420 mg.

Typically, the unit dose weight of the formulation can be about 320-400 mg. More typically, the unit dose weight of the formulation can be about 340-380 mg. Most typically, the unit dose weight of the formulation can be about 360-370 mg.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan ester selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a composition comprising:
(a) 50-65% w/w ibuprofen;
(b) 25-45% w/w One or more polyoxysorbitan esters;
(c) 3-8% w/w A hydroxide; and
(d) Water
   wherein the unit dose weight of the formulation can be about 600-840 mg.

Typically, The unit dose weight of the formulation can be about 640-800 mg. More typically, the unit dose weight of the formulation can be about 680-760 mg. Most preferably, the unit dose weight of the formulation can be about 720-740 mg.

Preferably, the composition can comprise:
(a) 50-60% w/w ibuprofen;
(b) 35-40% w/w One or more polyoxysorbitan esters;
(c) 3-8% w/w A hydroxide; and
(d) Water.

The composition can comprise:
(a) 50-65% w/w ibuprofen;
(b) 5-35% w/w One or more polyoxysorbitan esters;
(c) 5-10% w/w A hydroxide;
(d) 5-25% w/w Polyethylene glycol; and
(e) Water.
wherein the unit dose weight of the formulation can be about 600-840 mg.

Typically, The unit dose weight of the formulation can be about 640-800 mg. More typically, the unit dose weight of the formulation can be about 680-760 mg. Most preferably, the unit dose weight of the formulation can be about 720-740 mg.

The composition can consist essentially of:
(a) 50-65% w/w ibuprofen;
(b) 25-40% w/w One or more polyoxysorbitan esters;
(c) 3-8% w/w A hydroxide; and
(d) Water
wherein the unit dose weight of the formulation can be about 600-840 mg.

Typically, The unit dose weight of the formulation can be about 640-800 mg. More typically, the unit dose weight of the formulation can be about 680-760 mg. Most preferably, the unit dose weight of the formulation can be about 720-740 mg.

Preferably the composition can consist essentially of:
(a) 52-55% w/w ibuprofen;
(b) 35-40% w/w One or more polyoxysorbitan esters;
(c) 3-5% w/w A hydroxide; and
(d) Water.

The composition can consist essentially of:
(a) 50-65% w/w ibuprofen;
(b) 5-35% w/w One or more polyoxysorbitan esters;
(c) 5-10% w/w A hydroxide;
(d) 5-25% w/w Polyethylene glycol; and
(e) Water
wherein the unit dose weight of the formulation can be about 600-840 mg.

Typically, The unit dose weight of the formulation can be about 640-800 mg. More typically, the unit dose weight of the formulation can be about 680-760 mg. Most preferably, the unit dose weight of the formulation can be about 720-740 mg.

The composition can consist essentially of:
(a) 50-55% w/w ibuprofen;
(b) 30-35% w/w One or more polyoxysorbitan esters;
(c) 5-8% w/w A hydroxide;
(d) 4-8% w/w Polyethylene glycol; and
(e) Water
wherein the unit dose weight of the formulation can be about 600-840 mg.

Typically, The unit dose weight of the formulation can be about 640-800 mg. More typically, the unit dose weight of the formulation can be about 680-760 mg. Most preferably, the unit dose weight of the formulation can be about 720-740 mg.

The composition can consist essentially of:
(a) 50-65% w/w ibuprofen;
(b) 25-40% w/w One or more polyoxysorbitan esters;
(c) 3-8% w/w A hydroxide; and
(d) Water
wherein the unit dose weight of the formulation can be about 600-840 mg.

Typically, The unit dose weight of the formulation can be about 640-800 mg. More typically, the unit dose weight of the formulation can be about 680-760 mg. Most preferably, the unit dose weight of the formulation can be about 720-740 mg.

Preferably, the composition can consist essentially of:
(a) 52-55% w/w ibuprofen;
(b) 35-40% w/w One or more polyoxysorbitan esters;
(c) 3-5% w/w A hydroxide; and
(d) Water.

The composition can consist essentially of:
(a) 50-65% w/w ibuprofen;
(b) 5-35% w/w One or more polyoxysorbitan esters;
(c) 5-10% w/w A hydroxide;
(d) 5-25% w/w Polyethylene glycol; and
(e) Water.
wherein the unit dose weight of the formulation can be about 600-840 mg.

Typically, The unit dose weight of the formulation can be about 640-800 mg. More typically, the unit dose weight of the formulation can be about 680-760 mg. Most preferably, the unit dose weight of the formulation can be about 720-740 mg.

The composition can consist essentially of:
(a) 50-55% w/w ibuprofen;
(b) 30-35% w/w One or more polyoxysorbitan esters;
(c) 5-8% w/w A hydroxide;
(d) 4-8% w/w Polyethylene glycol; and
(e) Water
wherein the unit dose weight of the formulation can be about 600-840 mg.

Typically, The unit dose weight of the formulation can be about 640-800 mg. More typically, the unit dose weight of the formulation can be about 680-760 mg. Most preferably, the unit dose weight of the formulation can be about 720-740 mg.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan ester selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a soft gelatin capsule which contains a composition as described in any of the previous aspects.

According to an aspect of the present invention there is provided a soft gelatin capsule comprising a composition containing ibuprofen as described in the previous aspects wherein the composition allows for less than 5% of the ibuprofen dose to be solubilised within 5 minutes of immersion of the soft gelatine capsule in simulated gastric fluid at a pH of 1.2.

Preferably the composition allows for at least 10% of the ibuprofen dose to be solubilised within 10 minutes of immersion of the soft gelatine capsule in simulated gastric fluid at a pH of 1.2. More preferably the composition allows for at least 10% of the ibuprofen dose to be solubilised within 5 minutes of immersion of the soft gelatine capsule in simulated gastric fluid at a pH of 1.2.

Preferably the composition allows for at least 15% of the ibuprofen dose to be solubilised within 10 minutes of immersion of the soft gelatine capsule in simulated gastric fluid at a pH of 1.2. More preferably the composition allows for at least 15% of the ibuprofen dose to be solubilised within 5 minutes of immersion of the soft gelatine capsule in simulated gastric fluid at a pH of 1.2.

According to an aspect of the present invention there is provided a soft gelatin capsule comprising a composition of ibuprofen wherein the composition allows for at least 5% of the ibuprofen dose to be solubilised within 5 minutes of immersion of the soft gelatine capsule in simulated gastric fluid at a pH of 1.2.

Preferably the composition allows for at least 10% of the ibuprofen dose to be solubilised within 10 minutes of immersion of the soft gelatine capsule in simulated gastric fluid at a pH of 1.2. More preferably the composition allows for at least 10% of the ibuprofen dose to be solubilised within 5 minutes of immersion of the soft gelatine capsule in simulated gastric fluid at a pH of 1.2.

Preferably the composition allows for at least 15% of the ibuprofen dose to be solubilised within 10 minutes of immersion of the soft gelatine capsule in simulated gastric fluid at a pH of 1.2. More preferably the composition allows for at least 15% of the ibuprofen dose to be solubilised within 5 minutes of immersion of the soft gelatine capsule in simulated gastric fluid at a pH of 1.2.

According to an aspect of the present invention there is provided a composition for a soft-gel capsule which comprises ibuprofen and a solvent system wherein the solvent system comprises:
a) from about 60 to about 90% w/w of one or more polyoxysorbitan esters;
b) from about 5 to about 25% w/w of potassium hydroxide; and
c) water.

Typically, the solvent system comprises:
a) from about 75 to about 85% w/w of one or more polyoxysorbitan esters;
b) from about 10 to about 15% w/w of potassium hydroxide; and
c) water.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

Typically, the unit dose weight of the formulation is about 300-420 mg. More typically, the unit dose weight of the formulation is about 320-400 mg. Even more typically, the unit dose weight of the formulation is about 340-380 mg. Most typically, the unit dose weight of the formulation is about 370 mg.

Typically, the composition has a release rate for ibuprofen of at least 5% in 5 mins. More typically, the composition has a release rate of at least 10% in 5 mins.

Preferably the composition allows for at least 5% of the ibuprofen dose to be solubilised within 5 minutes of immersion of the soft gelatine capsule in simulated gastric fluid at a pH of 1.2.

Preferably the composition allows for at least 10% of the ibuprofen dose to be solubilised within 10 minutes of immersion of the soft gelatine capsule in simulated gastric fluid at a pH of 1.2. More preferably the composition allows for at least 10% of the ibuprofen dose to be solubilised within 5 minutes of immersion of the soft gelatine capsule in simulated gastric fluid at a pH of 1.2.

Preferably the composition allows for at least 15% of the ibuprofen dose to be solubilised within 10 minutes of immersion of the soft gelatine capsule in simulated gastric fluid at a pH of 1.2. More preferably the composition allows for at least 15% of the ibuprofen dose to be solubilised within 5 minutes of immersion of the soft gelatine capsule in simulated gastric fluid at a pH of 1.2.

According to an aspect of the present invention there is provided a soft-gel capsule which contains a composition which can comprise ibuprofen and a solvent system wherein the solvent system comprises:
a) from about 60 to about 90% w/w of one or more polyoxysorbitan esters;
b) from about 5 to about 25% w/w of potassium hydroxide; and
c) water
   wherein the composition has a weight of 300-420 mg and wherein the composition has a release rate for ibuprofen of at least 5% in 5 mins.

Preferably the composition allows for at least 5% of the ibuprofen dose to be solubilised within 5 minutes of immersion of the soft gelatine capsule in simulated gastric fluid at a pH of 1.2.

Preferably the composition allows for at least 10% of the ibuprofen dose to be solubilised within 10 minutes of immersion of the soft gelatine capsule in simulated gastric fluid at a pH of 1.2. More preferably the composition allows for at least 10% of the ibuprofen dose to be solubilised within 5 minutes of immersion of the soft gelatine capsule in simulated gastric fluid at a pH of 1.2.

Preferably the composition allows for at least 15% of the ibuprofen dose to be solubilised within 10 minutes of immersion of the soft gelatine capsule in simulated gastric fluid at a pH of 1.2. More preferably the composition allows for at least 15% of the ibuprofen dose to be solubilised within 5 minutes of immersion of the soft gelatine capsule in simulated gastric fluid at a pH of 1.2.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

According to an aspect of the present invention there is provided a composition for a soft-gel capsule contains a composition which comprises ibuprofen and a solvent system wherein the solvent system comprises:
a) from about 4 to about 55% w/w polyethylene glycol;
b) from about 10 to about 60% w/w of one or more polyoxysorbitan esters;
c) from about 4 to about 20% w/w of potassium hydroxide;
d) and water.

Typically, the solvent system comprises:
a) from about 4 to about 8% w/w polyethylene glycol;
b) from about 15 to about 20% w/w of one or more polyoxysorbitan esters;
c) from about 4 to about 8% w/w of potassium hydroxide;
d) and water.

Typically, the unit dose weight of the composition is about 300-420 mg. More typically, the unit dose weight of the composition is about 320-400 mg. Even more typically, the unit dose weight of the composition is about 340-380 mg. Most typically, the unit dose weight of the composition is about 370 mg.

Typically, the composition has a release rate for ibuprofen of at least 5% in 5 mins. More typically, the composition has a release rate of at least 10% in 5 mins.

Preferably the composition allows for at least 5% of the ibuprofen dose to be solubilised within 5 minutes of immersion of the soft gelatine capsule in simulated gastric fluid at a pH of 1.2.

Preferably the composition allows for at least 10% of the ibuprofen dose to be solubilised within 10 minutes of immersion of the soft gelatine capsule in simulated gastric fluid at a pH of 1.2. More preferably the composition allows for at least 10% of the ibuprofen dose to be solubilised within 5 minutes of immersion of the soft gelatine capsule in simulated gastric fluid at a pH of 1.2.

Preferably the composition allows for at least 15% of the ibuprofen dose to be solubilised within 10 minutes of immersion of the soft gelatine capsule in simulated gastric fluid at a pH of 1.2. More preferably the composition allows for at least 15% of the ibuprofen dose to be solubilised within 5 minutes of immersion of the soft gelatine capsule in simulated gastric fluid at a pH of 1.2.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan ester selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a soft-gel capsule containing a composition which can comprise:
(a) 50-60% w/w ibuprofen;
(b) 25-35% w/w of one or more polyoxysorbitan esters;
(c) 4-6% w/w A hydroxide;
(d) 5-8% w/w Polyethylene glycol; and
(e) Water
   wherein the composition has a weight of 300-420 mg and wherein the composition has a release rate for ibuprofen of at least 5% in 5 mins.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan ester selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a soft-gel capsule containing a composition which can comprise:
(a) 50-60% w/w ibuprofen;
(b) 35-40% w/w of one or more polyoxysorbitan esters;
(c) 3-6% w/w a hydroxide; and
(d) Water
   wherein the composition has a weight of 300-420 mg and wherein the composition has a release rate for ibuprofen of at least 5% in 5 mins.

Preferably the composition allows for at least 5% of the ibuprofen dose to be solubilised within 5 minutes of immersion of the soft gelatine capsule in simulated gastric fluid at a pH of 1.2.

Preferably the composition allows for at least 10% of the ibuprofen dose to be solubilised within 10 minutes of immersion of the soft gelatine capsule in simulated gastric fluid at a pH of 1.2. More preferably the composition allows for at least 10% of the ibuprofen dose to be solubilised within 5 minutes of immersion of the soft gelatine capsule in simulated gastric fluid at a pH of 1.2.

Preferably the composition allows for at least 15% of the ibuprofen dose to be solubilised within 10 minutes of immersion of the soft gelatine capsule in simulated gastric fluid at a pH of 1.2. More preferably the composition allows for at least 15% of the ibuprofen dose to be solubilised within 5 minutes of immersion of the soft gelatine capsule in simulated gastric fluid at a pH of 1.2.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

As used herein the reference to the solubilisation of ibuprofen refers to the release and solubilisation of an amount of the ibuprofen dose in simulated gastric fluid. For example, when the composition allows for the release and solubilisation of 5% of the ibuprofen dose within 5 minutes this means that within 5 minutes of immersion in simulated gastric fluid 5% of the ibuprofen dose is in solution. For example, if the ibuprofen dose is 100 mg then 5 mg of the ibuprofen will have been both released from the capsule and solubilised. An additional amount may have been released but will not have been solubilised within the 5 minute period following immersion of the gelatin capsule.

As used herein the reference to weight ratios refers to the percentage weight of the components in the composition. For example, a composition having a 40% by weight of a first component and 40% by weight of a second component has a weight ratio of 1:1.

As used herein the reference to the unit dose weight of the formulation refers to weight of the composition that would be filled into the gelatine shell. The terms ‘formulation’ and ‘composition’ are used interchangeably and have the same meaning.

For the avoidance of doubt, in accordance with the present invention it is the compositions which are for encapsulation within a gelatin shell that comprise the ibuprofen, polyoxysorbitan ester, polyethylene glycol and base and not the gelatin shell itself.

For the avoidance of doubt, the compositions described in the present specification are in the form of a liquid.

Embodiments of the invention will now be described by way of example only with reference to the accompanying Figures in which:

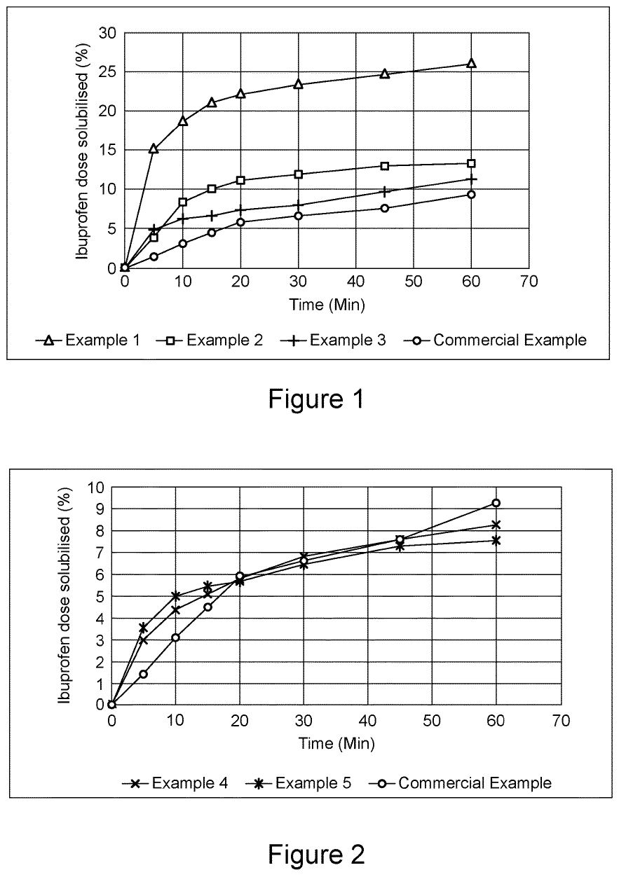

FIGS. 1 & 2 illustrate release profiles for ibuprofen from compositions of the present invention and current commercially available soft gelatin capsules.

Tables 1 & 2 illustrate examples of the composition of the present invention.

TABLE 1

| | Example 1 | Example 2 | Example 3 |
|---|---|---|---|
| Component | (% w/w) | (% w/w) | (% w/w) |
| Ibuprofen | 54.23 | 54.23 | 61.98 |
| Polysorbate 80 | 37.63 | 35.93 | 28.73 |
| KOH pellets | 4.88 | 5.90 | 5.58 |
| Purified water | 3.26 | 3.94 | 3.71 |
| Total | 100 | 100.00 | 100.00 |

TABLE 2

| Component | Example 4 (% w/w) | Example 5 (% w/w) |
|---|---|---|
| Ibuprofen | 54.23 | 61.98 |
| Polysorbate 80 | 30.57 | 20.63 |
| PEG 400 | 6.25 | 6.545 |
| KOH Pellets | 5.37 | 6.505 |
| Water | 3.58 | 4.34 |
| Total | 100.00 | 100.00 |

The formulation can be made using standard techniques known to the person of ordinary skill in the art. The polysorbate is initially heated to a temperature of 55° C. When polyethylene glycol is used the two materials are mixed together while heating to the same temperature. A portion of ibuprofen is added followed by a portion of aqueous potassium hydroxide solution while maintaining the heating. Once a homogenous solution has been achieved, the remaining ibuprofen and aqueous potassium hydroxide solution are added with continued heating. The resulting mixture is then stirred until a clear solution is obtained. The resulting solution can be stored until required for encapsulation in a gelatin capsule.

Similarly, the formulation can be encapsulated using standard soft gelatin encapsulation techniques well-known to the person skilled in the art. For example, the composition of the present invention can be deposited between two opposing ribbons of a gel composition. The composition of the ribbons may include gelatin and plasticisers such as glycerine and sorbitol and mixtures thereof. For example, a suitable gel composition can comprise about 35-50% by weight of gelatin, 20-40% by weight of a plasticiser and 25-50% by weight of water. The opposing ribbons are then run between two die rollers having die pockets thereon the surface of which corresponds to the configuration of the desired soft capsule. The composition is sealed within the fused casing.

The soft gelatin capsules were filled with compositions containing the standard dose of 200 mg of ibuprofen, i.e. in the examples above the amount of ibuprofen is 200 mg.

As can be seen from FIG. 1 and Table 3 below, the compositions of Examples 1-3 release the ibuprofen unexpectedly and significantly quicker than a current commercial product tested under the same conditions. As can be seen from FIG. 2 and Table 4 below, the compositions of Examples 4 and 5 release the ibuprofen at a rate that matches that of a current commercial example.

TABLE 3

| Time | Example 1 | Example 2 | Example 3 | Commercial Example |
|---|---|---|---|---|
| 0.0 | 0.0 | 0.0 | 0.0 | 0.0 |
| 5.0 | 14.8 | 3.9 | 4.9 | 1.43 |
| 10.0 | 18.6 | 8.3 | 6.3 | 3.1 |
| 15.0 | 21 | 10.1 | 6.6 | 4.5 |
| 20.0 | 22.1 | 11.2 | 7.4 | 5.87 |
| 30.0 | 23.3 | 11.9 | 8 | 6.63 |
| 45.0 | 24.6 | 13.0 | 9.7 | 7.6 |
| 60.0 | 26 | 13.3 | 11.3 | 9.23 |

TABLE 4

| Time | Example 4 | Example 5 | Commercial Example |
|---|---|---|---|
| 0.0 | 0.0 | 0.0 | 0.0 |
| 5.0 | 3.0 | 3.5 | 1.43 |
| 10.0 | 4.4 | 5.0 | 3.1 |
| 15.0 | 5.1 | 5.4 | 4.5 |
| 20.0 | 5.7 | 5.7 | 5.87 |
| 30.0 | 6.8 | 6.5 | 6.63 |
| 45.0 | 7.6 | 7.3 | 7.6 |
| 60.0 | 8.3 | 7.5 | 9.23 |

The release rate of the compositions of the present invention were determined by immersing the compositions in a gelatin capsule in simulated gastic fluid. The compositions were immersed into a beaker/container containing 900 ml of the simulated gastric fluid at a temperature of 37° C. The pH of the simulated gastric fluid was 1.2.

An advantage of the present invention is that there is provided a stable ibuprofen-containing composition which is suitable for encapsulation in a soft gelatin capsule and which releases the ibuprofen in a more solubilisable form than standard ibuprofen-containing soft gelatin capsules. A further advantage is provided with the ability to use a lower amount of base within the formulation whilst improving the solubilisation of the ibuprofen in simulated gastric fluid.

Further modifications can be made without departing from the scope of the invention described herein.

1-112. (canceled)

113. A composition for encapsulation in a soft gelatin shell, the composition comprising:
50%-65% w/w ibuprofen;
one or more polyoxysorbitan esters; and
a base,
wherein a weight ratio of the one or more polyoxysorbitan esters to the ibuprofen is from 0.1:1-0.9:1, a weight ratio of the base to the ibuprofen is from 1:6-1:20 and a weight ratio of the one or more polyoxysorbitan esters to the base is 1:1-13:1.

114. The composition as claimed in claim 113, wherein the weight ratio of the one or more polyoxysorbitan esters to the ibuprofen is 0.4:1-0.8:1.

115. The composition as claimed in claim 113, wherein the weight ratio of the one or more polyoxysorbitan esters to the ibuprofen is 0.5:1-0.7:1.

116. The composition as claimed in claim 113, wherein the weight ratio of the base to the ibuprofen is 1:8-1:15.

117. The composition as claimed in claim 116, wherein the weight ratio of the base to the ibuprofen is 1:9-1:11.5.

118. The composition as claimed in claim 113, wherein the weight ratio of the one or more polyoxysorbitan esters to the base is 3:1-9:1.

119. The composition as claimed in claim 118, wherein the weight ratio of the one or more polyoxysorbitan esters to the base is 5.5:1-7.75:1.

120. The composition as claimed in claim 113, wherein the weight ratio of the one or more polyoxysorbitan esters to the ibuprofen is 0.5:1-0.7:1, the weight ratio of the base to the ibuprofen is 1:9-1:11.5, and the weight ratio of the one or more polyoxysorbitan esters to the base is 5.5:1-7.75:1.

121. The composition as claimed in claim 113, wherein the composition comprises the ibuprofen, the one or more polyoxysorbitan esters, and the base,
wherein the weight ratio of the ibuprofen to the one or more polyoxysorbitan esters to the base is about 6-20:1-13:1.

122. The composition as claimed in claim 121, wherein the composition comprises the ibuprofen, the one or more polyoxysorbitan esters, and the base,
wherein the weight ratio of the ibuprofen to the one or more polyoxysorbitan esters to the base is about 9-12:5-8:1.

123. The composition as claimed in claim 113, wherein the composition further comprises water in an amount from about 2% w/w to about 10% w/w.

124. The composition as claimed in claim 113, wherein the composition comprises:
50-65% w/w of the ibuprofen;
30-40% w/w of the one or more polyoxysorbitan esters; and
3-10% w/w of a hydroxide,
wherein the weight ratio of the one or more polyoxysorbitan esters to the ibuprofen is 0.5:1-0.7:1, the weight ratio of the hydroxide to the ibuprofen is 1:9-1:11.5, and the weight ratio of the one or more polyoxysorbitan esters to the hydroxide is 5.5:1-7.75:1.

125. The composition as claimed in claim 124, wherein the composition comprises:
50-65% w/w of the ibuprofen;
30-40% w/w of the one or more polyoxysorbitan esters; and
4-7% w/w of the hydroxide,
wherein the weight ratio of the one or more polyoxysorbitan esters to ibuprofen is 0.5:1-0.7:1, the weight ratio of hydroxide to ibuprofen is 1:9-1:11.5, and the weight ratio of the one or more polyoxysorbitan esters to hydroxide is 5.5:1-7.75:1.

126. The composition as claimed in claim 113, wherein a unit dose weight of the composition is about 300-420 mg.

127. The composition as claimed in claim 126, wherein the unit dose weight of the composition is about 340-380 mg.

128. The composition as claimed in claim 113, wherein a unit dose of the composition has a fill volume of from 0.3 ml to 0.42 ml.

129. The composition as claimed in claim 113, wherein a unit dose weight of the composition is about 600-840 mg.

130. The composition as claimed in claim 129, wherein the unit dose weight of the formulation is about 680-760 mg.

131. The composition as claimed in claim 113, wherein a unit dose of the composition has a fill volume of from 0.6 ml to 0.84 ml.

132. A composition comprising:
50-65% w/w of ibuprofen;
25-45% w/w of one or more polyoxysorbitan esters;
3-8% w/w of a hydroxide; and
water,
wherein a volume of a unit dose of the composition is from about 0.3 ml to about 0.42 ml.

133. The composition as claimed in claim 132, wherein the composition consists essentially of:
52-55% w/w of the ibuprofen;
35-40% w/w of the one or more polyoxysorbitan esters;
3-5% w/w of the hydroxide; and
water,
wherein the volume of the unit dose of the composition is from about 0.3 ml to about 0.42 ml.

134. A composition comprising:
50-65% of w/w ibuprofen;
25-45% w/w of one or more polyoxysorbitan esters;
3-8% w/w of a hydroxide; and
water,
wherein a volume of a unit dose of the composition is from about 0.6 ml to about 0.84 ml.

135. The composition as claimed in claim 134, wherein the composition consists essentially of:
52-55% w/w of the ibuprofen;
35-40% w/w of the one or more polyoxysorbitan esters;
3-5% w/w of the hydroxide; and
water,
wherein the volume of the unit dose of the composition is from about 0.6 ml to about 0.84 ml.

136. The composition as claimed in claim 113, wherein the composition has a release rate for ibuprofen of at least 5% in 5 minutes.

137. The composition as claimed in claim 136, wherein the composition has a release rate of at least 10% in 5 minutes.

138. A soft gelatin capsule which contains a composition as claimed in claim 113.

139. The soft gelatin capsule as claimed in claim 138, wherein the composition allows for at least 5% of the ibuprofen to be solubilized within 5 minutes of immersion of the soft gelatin capsule in simulated gastric fluid at a pH of 1.2.

140. The soft gelatin capsule as claimed in claim 138, wherein the composition allows for at least 15% of the ibuprofen to be solubilized within 5 minutes of immersion of the soft gelatin capsule in simulated gastric fluid at a pH of 1.2.

141. The composition as claimed in claim 113, wherein the polyoxysorbitan ester is selected from polysorbate 20, polysorbate 21, polysorbate 40, polysorbate 60, polysorbate 61, polysorbate 65, polysorbate 80, polysorbate 81, polysorbate 85, and polysorbate 120.

142. A composition for a soft-gel capsule, the composition comprising:
ibuprofen; and
a solvent system, the solvent system comprising:
from about 60 to about 90% w/w of one or more polyoxysorbitan esters;
from about 5 to about 25% w/w of potassium hydroxide; and
water.

143. The composition as claimed in claim 142, wherein the solvent system comprises:
from about 75 to about 85% w/w of the one or more polyoxysorbitan esters;
from about 10 to about 15% w/w of the potassium hydroxide; and
water.
